(19) United States
(12) Patent Application Publication
(10) Pub. No.: US 2023/0190630 A1
(43) Pub. Date: Jun. 22, 2023

(54) Jet Fuel Wash

(71) Applicant: Kevin Miller, Chattanooga, TN (US)

(72) Inventor: Kevin Miller, Chattanooga, TN (US)

(21) Appl. No.: 17/553,015

(22) Filed: Dec. 16, 2021

(51) Int. Cl. A61K 8/92; A61K 8/02

(52) U.S. Cl. CPC A61K 8/922; A61K 8/0208; A61Q 19/10

(57) ABSTRACT

A specific recipe for soap that eliminates the extremely stubborn smell of jet fuel or other hydrocarbon liquid.

### CROSS-REFERENCES TO RELATED APPLICATIONS

Not Applicable.

### FIELD OF INVENTION

The present invention relates generally to a cleaning product. More specifically, the present invention pertains to a soap for use at least on skin and cloth materials.

### BACKGROUND OF THE INVENTION

Jet fuel was engineered to smell horrible and not dissipate. The wretched smell will take days to dissipate from a person's skin, and even longer for one's hair. When it gets on your clothes, you have to leave them outside for more than a week. It may take more than a week for the smell to dissipate from clothing and washing them with other clothes may cause the smell to infected the other clothes. It may require one's clothes to be left outside until the smell has dissipated. Conventional cleaning products do little to resolve the problem.

Additionally, prolonged exposure of jet fuel to skin can be harmful and effective removal of the substance is needed as a safety precaution for people that handle the substance or a regular basis. No product exists to solve this problem.

There exists a need for a cleaning product that is safe to use on skin but strong enough to remove any trace of pungently aromatic hydrocarbons.

### SUMMARY OF THE INVENTION

This Summary is provided to introduce a selection of concepts in a simplified form that is further described below in the Detailed Description. This Summary is not intended to identify key features or essential features of the claimed subject matter, nor is it intended to be used as an aid in determining the scope of the claimed subject matter. The present invention is defined by the claims.

The present invention is directed towards a specific recipe for soap that eliminates the extremely stubborn smell of jet fuel. Some embodiments of the present invention may comprise a base liquid soap made for scale production mix with natural essential oils that are not commonly used in soap that are combined in the right percentages such that they are completely skin safe.

### BRIEF DESCRIPTION OF THE DRAWINGS

The present invention is described in detail below with reference to the attached drawing figures, wherein:

FIG. 1 illustrates a flowchart providing an exemplary method in accordance with aspects of the present invention.

### DETAILED DESCRIPTION OF THE INVENTION

Some embodiments of the present invention are described with specificity herein to meet statutory requirements. However, the scope of the invention is not intended to be defined by the description itself. The claimed subject matter may be embodied as to include different features, elements, components, steps, or combinations of steps, similar to those described herein, and in combination with other existing or future technologies. Moreover, although the term “step” might be used to connote different elements of the methods employed, this term should not be interpreted as implying any particular order among or between various steps herein disclosed unless and except the order of individual steps is explicitly described or required.

The present invention is directed towards a specific recipe for soap that eliminates the extremely stubborn smell of jet fuel. Some embodiments of the present invention may comprise a base liquid soap made for scale production mix with natural essential oils that are not commonly used in soap that are combined in the right percentages such that they are completely skin safe.

Some embodiments of the present invention may be produced by combining 15 ounces of base liquid soap, 1 ml of citronella oil, 0.25 ml of peppermint oil, 0.5 ml of Eucalyptus oil, 0.5 ml of sandalwood oil, 0.5 ml of lemon oil, and 50 grams of abrasive material. Abrasive material may include, but not be limited to, pumice, pulverize or powdered nut shells, such as almond shells or walnut shells, other cellulose-based exfoliants and the like or combinations thereof. One having skill in the art will recognize that concentrations of more than 3% essential oils are not safe for continuous use directly on skill. As such, formulations of the present invention for direct use on skin include no more than 3% total essential oil by volume.

Some embodiments of the present invention may include shower soap, shampoo, laundry detergent and the like. Some embodiments of the present invention may include handwipes which are highly portable and can be kept near a mechanics workspace for use immediately after exposure to jet fuel or other hydrocarbon liquid. Such a handwipe may comprise a liquid absorbent and disposable hand tower that may be saturated with the cleaning product described herein. Such a disposable hand tower may comprise paper, cotton, woven bamboo fiber, or the like or combinations thereof.

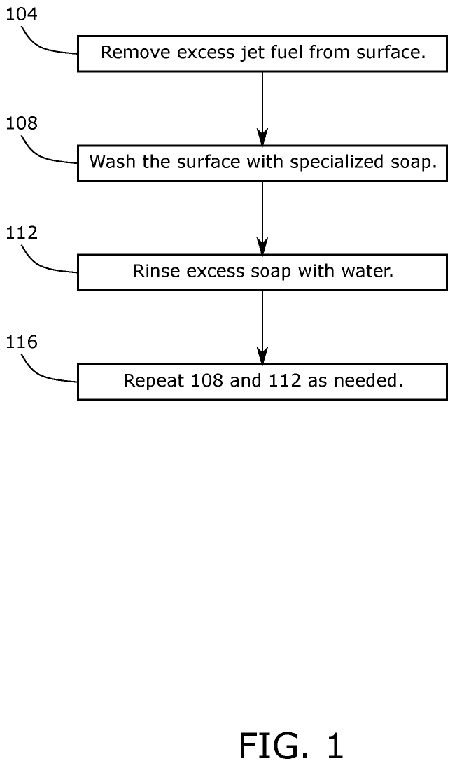

FIG. 1 illustrates a flowchart 100 providing an exemplary method in accordance with aspects of the present invention. At step 104, excess jet fuel and/or other hydrocarbon liquid, if any, is removed from the surface. As used herein, “surface” is a broad term that includes, but is not limited to floors, countertops, parts, tools, clothing, hair, hands, skin, or the like. At step 108, washing the surface affected by the hydrocarbon liquid with a specialize soap comprising base liquid soap, citronella oil, peppermint oil, eucalyptus oil, sandalwood oil, lemon oil, and abrasive material. At step 112, the excess soap may be rinsed from the surface with water. At step 116, steps 108 through 112 may be repeated as needed.

Embodiments of the present invention have been described, as required by statute, to be illustrative, but should not be interpreted to be restrictive. One having skill in the art will recognize that many different arrangements of the various components depicted are possible without departing from the scope of the claims below, as well as arrangements including components not explicitly shown.

One having skill in the art will understand that certain combinations and/or sub-combinations of elements and features are of utility and may be employed without reference to other combinations and/or sub-combinations and are contemplated within the scope of the claims. Not all steps listed in the various figures need be carried out in the specific order described.

What is claimed is:

1. A composition of matter for removing hydrocarbon liquids from a surface comprising:
citronella oil;
peppermint oil;
eucalyptus oil;
sandalwood oil; and
lemon oil.

2. The composition of matter of claim 1, further comprising:
a base liquid soap and abrasive material for use on hands and skin.

3. The composition of matter of claim 1, further comprising:
a liquid absorbent disposable towel.

4. A method for removing hydrocarbon liquids from a surface, comprising:
washing the surface affected by the hydrocarbon liquid with a specialize soap, wherein the specialize soap comprises:
base liquid soap;
citronella oil;
peppermint oil;
eucalyptus oil;
sandalwood oil;
lemon oil; and
abrasive material.
